(19) United States
(12) Patent Application Publication
(10) Pub. No.: US 2015/0018418 A1
(43) Pub. Date: Jan. 15, 2015

(54) COMPOSITIONS AND METHODS FOR PROLONGING PREGNANCY

(71) Applicant: Thomas Guttuso, Jr., Snyder, NY (US)

(72) Inventor: Thomas Guttuso, Jr., Snyder, NY (US)

(21) Appl. No.: 14/324,338

(22) Filed: Jul. 7, 2014

Related U.S. Application Data
(60) Provisional application No. 61/957,645, filed on Jul. 9, 2013.

(51) Int. Cl. A61K 31/197

(52) U.S. Cl. CPC A61K 31/197
USPC 514/561

(57) ABSTRACT

Provided are compositions and methods for use for prolonging pregnancy. The methods involve administering to an individual in need thereof an effective amount of a composition that contains a compound which is capable of specifically binding to an alpha2delta subunit of a voltage-gated calcium channel, such as gabapentin, pregabalin, and pharmaceutically acceptable salts and prodrugs thereof The composition is administered such that pregnancy is prolonged to by reducing, stopping, preventing and/or preventing recurrence of preterm labor during a human pregnancy. Also provides are articles of manufacture that have packaging containing one or a combination of compounds that are capable of specifically binding an alpha2delta subunit of a voltage-gated calcium channel. The compound is provided in an amount sufficient for alleviating and/or preventing preterm labor in a pregnant human female. The packaging comprises printed material, the printed material providing an indication that the pharmaceutical composition is for use in the alleviating or preventing of preterm labor and/or for prolonging pregnancy.

### CROSS REFERENCE TO RELATED APPLICATIONS

This application claims priority to U.S. provisional application no. 61/957,645, filed Jul. 9, 2013, the disclosure of which is incorporated herein by reference.

### FIELD

The present disclosure relates generally to reproductive health and more specifically to methods of prolonging pregnancy.

### BACKGROUND

Preterm labor (PTL) precedes 40-50% of premature births, which is defined as birth occurring before 37 weeks gestation. Premature birth occurs in 11.5% of all live births in the US, is the number one cause of neonatal mortality, and is responsible for half of the long-term neurological impairments in children. Premature birth accounts for 35% of all health care spending for infants in the US. The incidence of premature birth has remained fairly constant over the past 40 years despite the use of multiple different management methods. For example, tocolytics such as intravenous (i.v.) administration of magnesium sulfate or oral or i.v. administration of nifedipine or terbutaline are effective in arresting the uterine contractions of PTL; however, they prolong pregnancy for only about 2-7 days. This may provide the time needed to administer steroids or transfer the patient to a facility with a neonatal intensive care unit but there does not appear to be any long-term benefit to the fetus from prolonging pregnancy for this short of a period of time. Further, maintenance tocolysis with nifedipine or terbutaline in patients with recent PTL has been shown to be no more effective than maintenance placebo therapy for prolonging pregnancy and, therefore, maintenance tocolysis is currently not recommended. Thus, there is an ongoing and unmet need for improved approaches for prolonging pregnancy in female individuals who are either undergoing PTL, recently had PTL or are at risk for recurrent PTL during a single pregnancy. The present disclosure meets these needs.

### SUMMARY

The present disclosure provides methods that are suitable for prolonging pregnancy. In general, the methods comprise administering to an individual in need thereof an effective amount of a composition comprising a compound which is capable of specifically binding to an alpha2delta subunit of a voltage-gated calcium channel. Such compounds are known in the art and include but are not necessarily limited to gabapentin, pregabalin, PD 200-390, PD 332-334, PD 299-685, PF-05089771, PF-05212377 (SAM-760), PF-06305591 or PF-06273340, and combinations thereof In certain embodiments, the compound is a gastroretentive form of gabapentin or is gabapentin enacarbil. In embodiments, the composition is administered in an amount ranging from 10 to about 5000 mg per day, and can be administered over a series of days so that in a patient with active or recent preterm labor the pregnancy can be prolonged preferably to more than 37 weeks of gestation, which is considered full term.

In certain embodiments, the individual in need of treatment is at risk for delivering a premature baby due to any of a variety of circumstances which include but are not necessarily limited to PTL, cervical incompetence or insufficiency, a short cervix, conception through in-vitro fertilization, and/or a multiple birth pregnancy. In one embodiment, the individual in need of treatment is experiencing PTL, or is not experiencing PTL but previously had PTL during the same pregnancy.

In another aspect the disclosure provides an article of manufacture comprising packaging containing a pharmaceutical composition comprising one or a combination of the aforementioned compounds that are capable of specifically binding an alpha2delta subunit of a voltage-gated calcium channel. The compound is provided in an amount sufficient for alleviating and/or preventing preterm labor in a pregnant human female. The packaging comprises printed material, the printed material providing an indication that the pharmaceutical composition is for use in the alleviating or preventing preterm labor or prolonging pregnancy in a women at risk for premature birth.

### DETAILED DESCRIPTION

The present disclosure provides compositions and methods suitable for prolonging pregnancy. One aspect of the invention comprises administering to an individual in need thereof an effective amount of a composition comprising a compound which specifically binds to an alpha2delta subunit of a voltage-gated calcium channel. In an embodiment, the individual in need of treatment according to the present disclosure is at risk for delivering a premature baby due to PTL, cervical incompetence or insufficiency, a short cervix (less than 30 millimeters), conception through in-vitro fertilization, and/or a multiple birth pregnancy. In one embodiment, the individual in need of treatment is undergoing PTL. In another embodiment, the individual in need of treatment is a pregnant human female who is not experiencing PTL, but previously experienced PTL during the same pregnancy. PTL is generally defined as having regular uterine contractions of at least 5/hour with cervical dilation or progressive cervical change in a pregnant woman before 37 weeks of gestation. In embodiments, PTL does not include a miscarriage. In embodiments, pregnancy is prolonged, thereby demonstrating administration of an effective amount of a compound as described herein by preventing PTL or its recurrence during a pregnancy, or by reducing the severity and/or duration of PTL such that premature birth does not occur. Premature birth as used herein is a considered to be the live birth of a human newborn before 37 weeks of gestation.

Compounds which specifically bind to an alpha2delta subunit of a voltage-gated calcium channel and are suitable for use in the present invention are known in the art and include but are not necessarily limited to analogs of y-Aminobutyric acid (“GABA”) and pharmaceutically acceptable salts thereof In embodiments, the GABA analog is selected from those described in U.S. Pat. No. 6,310,098, and from which the description of the GABA analogs is incorporated herein by reference. In embodiments, the GABA analog is selected from the group consisting of gabapentin, pregabalin, PD 200-390, PD 332-334 or PD 299-685 (known to have specific alpha2delta binding activity) or PF-05089771, PF-05212377 (SAM-760), PF-06305591 or PF-06273340 (the latter group having potential exhibit specific alpha2delta binding activity), and combinations thereof In one embodiment, the compound is gabapentin. In another embodiment, the compound is pregabalin.

As is well known in the art, gabapentin is (1-aminomethyl)cyclohexaneacetic acid. It has a molecular formula of C₉H₁₇NO₂ and a molecular weight of 171.24 g/mol. The structural formula of gabapentin is:

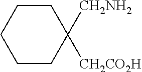

Compositions comprising gabapentin as an active ingredient are sold under several trade names, including NEURONIN, HORIZANT (gabapentin enacarbil, an extended release gabapentin prodrug tablet form), and GRALISE (a film-coated gastroretentive tablet formulation).

Gabapentin encarbil has the molecular formula of C₁₆H₂₇NO₆, a molecular weight of 329.389 g/mol. It has the structure:

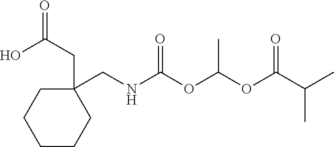 [SMILES: CC(OC(=O)NCC1(CC(=O)O)CCCCC1)OC(=O)C(C)C]

As is well known in the art, pregablin is (S)-(3-aminomethyl)-5-methylhexanoic acid, or (S)-isobutyl GABA. It has a molecular formula of C₈H₁₇NO₂ and a molecular weight of 159.23 g/mol. The structural formula of pregabalin is:

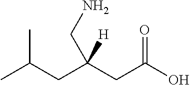

Compositions comprising pregablin as an active ingredient are sold under several trade names, including LYRICA and NERVALIN.

Compositions comprising gabapentin or pregabalin are described in U.S. Pat. Nos. 6,818,787, 8,026,279, 8,048,917, 6,340,475, and 6,488,962, and the descriptions of these compounds and pharmaceutical compositions comprising in these patents are incorporated herein by reference.

Dosing of the compositions comprising a compound which specifically binds to an alpha2delta subunit of a voltage-gated calcium channel in order to prolong pregnancy can be determined in conjunction with the knowledge of the skilled artisan, given the benefit of the present disclosure. In embodiments, the age, general and/or reproductive health, cervical condition, number of pregnancies and history of pregnancy complications/miscarriages, as well as the health and number of fetuses the individual is carrying, may be taken into account when determining an effective amount and dosing regimen. In embodiments the compound is administered in an amount of about 10 mg to about 5000 mg a day, inclusive, and including all integers and ranges there between. In one embodiment, the compound is administered such that PTL is stopped. In another embodiment, the compound is administered at least once daily until a physician or other health care provider makes a determination that the risk of a recurrent episode of PTL has been satisfactorily reduced. In one embodiment, the compound is administered at least once per day such that PTL is prevented for at least 3, 4, 5, 6, or 7 days, or longer. In embodiments the compound is administered at least once per day such that PTL is prevented until at least 30, 31, 32, 33, 34, 35, 36, or 37 weeks of gestation is achieved.

The route of administration can be any suitable route. In embodiments, the composition comprising the compound which specifically binds to an alpha2delta subunit of a voltage-gated calcium channel is administered orally, parenterally, subcutaneously, intravenously, intramuscularly, intraperitoneally, transdermally, by intranasal instillation, by implantation, by intracavitary or intravesical instillation, intraocularly, intraarterially, intralesionally, or by application to mucous membranes.

The form of pharmaceutical preparation is also not particularly limited. In embodiments, the compound is present in a pharmaceutical composition comprising the compound, and/or a prodrug or comprising the compound and a pharmaceutically-acceptable carrier, diluent or excipient, and suitable such components are well known in the art. Some examples of such carriers, diluents and excipients can be found in: Remington: The Science and Practice of Pharmacy (2005) 21st Edition, Philadelphia, PA. Lippincott Williams & Wilkins. In embodiments the pharmaceutical composition can be provided in an aerosolized, liquid or solid dosage form. Solid dosage forms include but are not necessarily limited to tablets, capsules, caplets, and strips, for swallowing or oral dissolution, and may be provided for rapid or extended release.

In another aspect, the invention provides a method for selecting an individual as a candidate for a therapy designed to prolong pregnancy. This approach comprises determining that an individual has been pregnant for less than 37 weeks and that the individual is experiencing PTL, or determining that the individual experienced PTL during the pregnancy, and selecting the individual as a candidate for pregnancy prolongation as described herein. In an embodiment, the method further comprises administering to the individual a compound which specifically binds to an alpha2delta subunit of a voltage-gated calcium channel in order to prolong the pregnancy.

In another aspect the disclosure includes an article of manufacture which contains packaging, wherein the packaging contains a pharmaceutical formulation comprising a compound which specifically binds to an alpha2delta voltage-gated calcium channel. The packaging may comprise a container that is separate from the packaging, or the packaging can itself be a container. In embodiments, the packaging or container is a closed or sealed vial, a bottle, a blister (bubble) pack, or any other suitable container for the sale, or distribution, or use of pharmaceutical agents. The packaging can comprise any suitable packaging material, such as plastic or cardboard, and can be provided as in any conventional form or shape, including but not necessarily limited to a paper or cardboard box. In addition to the pharmaceutical compositions, the package may contain printed information. The printed information can be provided on a label, or on a paper insert, or printed on the packaging material itself. The printed information can include information that identifies the amounts and types of active and inactive ingredients in the package, an indication of what condition the pharmaceutical composition(s) is intended to treat, and instructions for taking the pharmaceutical composition, such as the number of doses to take or to administer over a given period of time. In embodiments, the article of manufacture includes printed material that provides an indication that it contains gabapentin or pregablin or pharmaceutical salts thereof, or any other compound disclosed herein, and that the formulation is to be used to prolong pregnancy, and/or to stop PTL, or to prevent recurrence of PTL during a pregnancy.

The following Examples are intended to illustrate embodiments of the invention but are not intended to be limiting.

### EXAMPLE 1

A 19 year-old pregnant white female, at 29.5 weeks gestation, presented to the hospital in PTL during her first pregnancy. The patient had a past medical history significant for supraventricular tachycardia controlled with atenolol 50 mg a day and mild cerebral palsy since birth with normal intelligence and mild spastic gait. The patient's mother experienced PTL during one of her pregnancies, leading to premature birth of the patient at 30 weeks gestation. The patient's sister also had experienced PTL with one of her pregnancies resulting in premature birth at 36 weeks gestation.

On presentation, the patient was found to be having regular uterine contractions, each lasting at least 1 minute at a rate of 10 contractions/hour. The initial pelvic examination showed intact fetal membranes and cervical dilatation of 1 cm and 50% cervical effacement. A repeat pelvic exam 6 hours later showed the cervix had progressively dilated to 2.5 cm and remained 50% effaced. The uterine contractions remained at a rate of 10/hour.

The patient was diagnosed with PTL and started on nifedipine 20 mg by mouth every 6 hours for tocolysis and magnesium sulfate 6 g as an intravenous infusion over 30 minutes followed by 2 g per hour for the next 12 hours for fetal neuroprotection. The uterine contractions slowly diminished and were completely resolved 12 hours after tocolysis was initiated. A course of steroids were also given for fetal lung maturation.

The patient then enrolled in a study conducted in advancement of the present disclosure and began therapy with gabapentin 300 mg, 3×/day (900 mg/day total). The first gabapentin dose was administered 20 hours after tocolysis with nifedipine was initiated and 8 hours after uterine contractions had fully resolved. At the time of enrollment, the pelvic exam findings were unchanged, showing cervical dilation of 2.5 cm and effacement of 50%. The patient experienced mild dizziness side effects after the first dose, which resolved that same day. The following day the patient was discharged home only on gabapentin 300 mg, 3×/day and her previous outpatient dose of atenolol at 50 mg a day. Five days after enrolling in the study, the patient reported feeling uterine cramping and contractions identical to what she had experienced before being initially diagnosed with PTL. She was instructed to increased her gabapentin dose to 600 mg, 3×/day. After the second 600 mg gabapentin dose was taken, the patient reported that all uterine cramping and uterine contraction feelings fully resolved. She did not experience any side effects from the higher gabapentin dose.

The patient remained stable for the following 16 days on 600 mg of gabapentin, 3×/day, without any uterine cramping, after which the cramping recurred. She returned to the hospital on the same day and was found to be having uterine contractions, each lasting about 15-20 seconds, occurring every 4 minutes. Pelvic exam at that time showed the cervix to still be 2.5 cm dilated and 50% effaced. The patient was started on an intravenous magnesium sulfate infusion of 2 g/hour and within 5 hours all uterine contractions had resolved. Gabapentin was not administered while the patient was in the hospital. Fetal monitoring throughout this hospitalization was reassuring and without concern. The patient was discharged. However, the patient apparently misunderstood the discharge instructions given and did not resume the gabapentin therapy. Eight days after discontinuing the gabapentin therapy, the patient reported that she was no longer feeling spontaneous fetal movement. She did not alert any family members or any of her doctors until she continued to not feel any fetal movements for four more days, upon which she presented to the hospital and was found to have intrauterine fetal death (IUFD). The fetus was delivered the following day and no cause of fetal demise was determined An autopsy was not performed on the fetus. A fetal tissue sample that was obtained for genetic testing was found to be inadequate and genetic testing was not performed. It should be noted that the serum half-life of gabapentin is 6 hours in humans. Therefore, gabapentin is expected to be fully cleared about 30 hours after its discontinuation.

This case is believed to provide the first evidence associating gabapentin maintenance therapy with prolonging pregnancy and fetal survival in a patient with PTL and illustrates at least two aspects of the invention: 1) Increasing the gabapentin dose from 300 mg to 600 mg, 3×/day, led to the rapid resolution of the patient's recurrent premature uterine contractions. Such contractions are the hallmark of preterm labor, which is known to result in premature birth in 60% of cases. 2) The discontinuation of maintenance gabapentin therapy for 8 days in this patient with preterm labor was associated with the IUFD. It is not clear how gabapentin maintenance therapy could have been preserving fetal survival in this case. Nevertheless, the association of discontinuing gabapentin and IUFD is strong in this case as formal fetal monitoring obtained in the hospital was normal for the first 24 hours after gabapentin was discontinued, a time period when serum gabapentin would still be expected to be present. However, fetal movements were no longer apparent 7 days later when gabapentin would have been fully cleared for at least 6 days. Thus, this Example demonstrates for the first time the apparent capability of gabapentin therapy administered subsequent to a PTL episode to alleviate the PTL and accordingly prolong the pregnancy during which the gabapentin was administered.

### EXAMPLE 2

A 22 year-old pregnant white female was found to be in PTL at 31 weeks gestation. The cervix was dilated at 3.5-4 cm and regular uterine contractions were occurring at 11/hour. The patient was started on nifedipine 20 mg by mouth every 6 hours for tocolysis and magnesium sulfate 6 g as an intravenous infusion over 30 minutes followed by 2 g per hour for the next 12 hours for fetal neuroprotection. The uterine contractions slowly diminished and were completely resolved 14 hours after tocolysis was initiated. A course of steroids were also given for fetal lung maturation.

The next day, the patient enrolled in a study conducted in advancement of the present disclosure and began therapy with gabapentin 300 mg, 3×/day (900 mg/day total). The cervix was still 3.5-4 cm dilated at the time of enrollment. By the evening of the next day, the subject began feeling “uncomfortable” with a “pressure” feeling in her uterus. She was instructed the next day to increase her bedtime gabapentin dose to 600 mg and to continue 300 mg in the morning and in the afternoon. The following morning the subject reported that she felt normal and the uncomfortable pressure feeling in her uterus had fully resolved. Two days later the subject reported feeling some recurrent uterine pressure especially noticeable for the hour before each gabapentin dose was taken. Gabapentin was increased to 600 mg, 3×/day and by the following day all of the pressure symptoms had resolved. The subject remained asymptomatic.

Two days later the patient had an appointment with her obstetrician, which showed her cervix to be 4 cm dilated and the baby's heart rate at 135 beats/minute. She continued on gabapentin 600 mg, 3×/day and remained asymptomatic. Three days later the subject noticed that her “water broke”. She was admitted to the hospital and confirmed to have preterm premature rupture of membranes (PPROM) with cervix still dilated to 4 cm. Gabapentin was continued at 600 mg, 3×/day. The subject remained asymptomatic throughout the next day; however, on the morning of the following day the subject began having regular uterine contractions about every 3-5 minutes and her cervix had now dilated to 7cm. Fetal ultrasound showed the baby to be breach in the uterus and, therefore, the subject was taken for caesarian section at 33, 1/7 weeks gestation.

As was seen in Example 1, Example 2 also demonstrated, on two occasions, that recurrent uterine pressure symptoms were fully resolved after increasing gabapentin dosing leading to a prolongation of pregnancy. This discovery of gabapentin maintenance therapy resolving recurrent uterine contractions and pressure symptoms leading to a prolongation of pregnancy and preserving fetal survival is accordingly highly valuable to the public. Its value can be recognized at least in part because uterine contractions are an essential component of PTL, which precedes 40-50% of premature births (<37 weeks gestation) and as described above, premature birth occurs in 11.5% of all live births in the US, is the number one cause of neonatal mortality, and is responsible for half of the long-term neurological impairments in children. Premature birth also accounts for 35% of all health care spending for infants in the US, and its incidence has remained fairly constant over the past 40 years despite the use of multiple different management methods.

It will be recognized by those skilled in the art from the foregoing disclosure and Examples presented herein that the discovery that the use of a compound which specifically binds to an alpha2delta subunit of a voltage-gated calcium channel to prolong pregnancy is surprising. For instance, while there are no prior reports of gabapentin therapy being associated with prolonging pregnancy, there is at least one report that discloses use of gabapentin during pregnancy is associated with an increase in premature birth. (Fujii H, et al. Neurology 2013;80:1565-70.) Further, the mechanism of action of gabapentin and related GABA analogs does not provide a basis for expecting that such compounds would be suitable for prolonging pregnancy. In particular, gabapentin was FDA-approved in 1993 for the treatment of seizures, in 2002 for postherpetic neuralgia and in 2011 for restless legs syndrome, none of which are related to the duration of pregnancy. Moreover, as discussed above, the principle binding site in the nervous system for gabapentin is the alpha-2/delta subunit of L-type voltage-gated calcium channels (VGCCs) and gabapentin's mechanism of action likely involves calcium channel antagonism. Nifedipine is a calcium channel antagonist active at a different subunit of the VGCC (the alpha-1 subunit) and while it is a commonly used tocolytic, it prolongs pregnancy for only about 2-5 days, which does not create a basis for predicting how a compound such as gabapentin would function during PTL, nor does it provide a suggestion that such compounds should be tested for such a function. In fact, maintenance therapy with nifedipine has been shown in two separate studies to provide no prolongation of pregnancy in patients with resolved PTL (Lyell D J, et al. Obstet Gynecol 2008;112:1221-6; Roos C, et al. Jama 2013;309:41-7), which is in contrast to the results presented in the Examples of this disclosure using gabapentin as a representative GABA analog. Furthermore, there is no evidence to support the use of maintenance tocolytics for prolonging pregnancy or resuming tocolytic therapy for recurrent PTL, and thus neither practice is currently recommended. Therefore, there are no prior data showing gabapentin use in pregnancy, nor are there data on the use of other calcium channel antagonists which suggest that maintenance therapy with these classes of drugs could prolong pregnancy. Accordingly, the present disclosure provides a long needed solution to reducing PTL, and thus prolonging pregnancies during which PTL occurs, and is at risk for recurrence.

While the invention has been described through specific embodiments, routine modifications will be apparent to those skilled in the art and such modifications are intended to be within the scope of the present invention.

What is claimed is:

1. A method for prolonging pregnancy in a pregnant human female experiencing preterm labor or at risk for recurrence of a subsequent preterm labor during the pregnancy, the method comprising administering to the pregnant human female an effective amount of a composition comprising an active ingredient, wherein the active ingredient is selected from gabapentin, pregabalin, and pharmaceutically acceptable salts or prodrugs thereof

2. The method of claim 1, wherein the active ingredient is gabapentin or a pharmaceutically acceptable salt or prodrug thereof

3. The method of claim 2, wherein the active ingredient is administered in an amount of about 10 to about 5000 mg per day.

4. The method of claim 2, wherein the active ingredient comprises a gabapentin prodrug, and/or wherein the composition comprises gabapentin in a gastroretentive formulation.

5. The method of claim 4, wherein the gabapentin prodrug is gabapentin enacarbil.

6. The method of claim 3, wherein the active ingredient is administered in an amount of from 900 mg to 1800 mg per day for a period of at least two days.

7. A method of prolonging pregnancy in a pregnant patient at risk for delivering a premature baby, the method comprising administering to the pregnant patient a composition comprising a compound which binds an alpha2delta subunit of a voltage-gated calcium channel such that the pregnant patient does not deliver the baby prematurely.

8. The method of claim 7, wherein the patient is at risk for delivering a premature baby due to preterm labor, cervical incompetence or insufficiency, a cervix of less than 30 millimeters, conception through in-vitro fertilization, or a multiple birth pregnancy.

9. An article of manufacture comprising packaging containing a pharmaceutical composition comprising a compound which is capable of specifically binding an alpha2delta subunit of a voltage-gated calcium channel in an amount sufficient for alleviating and/or preventing preterm labor in a pregnant human female, the packaging comprising printed material, the printed material providing an indication that the pharmaceutical composition is for use in the alleviating or preventing preterm labor.

10. The article of manufacture of claim 9, wherein the compound is selected from gabapentin or pregabalin, or a pharmaceutically acceptable salt or prodrug thereof

11. The article of manufacture of claim 10, wherein the compound is a gabapentin prodrug, and/or wherein the compound is gabapentin and is provided in a gastroretentive formulation.

12. The article of manufacture of claim 11, wherein gabapentin prodrug is gabapentin enacarbil.
